(19) United States
(12) Patent Application Publication
(10) Pub. No.: US 2017/0112390 A1
(43) Pub. Date: Apr. 27, 2017

(54) ATRIAL ARRYTHMIA DETECTION USING A PRESSURE SIGNAL IN AN IMPLANTABLE MEDICAL DEVICE AND MEDICAL SYSTEM

(71) Applicant: Medtronic, Inc., Minneapolis, MN (US)

(72) Inventors: Yong K. Cho, Excelsior, MN (US); Michael R.S. Hill, Minneapolis, MN (US)

(21) Appl. No.: 14/919,854

(22) Filed: Oct. 22, 2015

(51) Int. Cl. A61B 5/0215; A61N 1/365; A61N 1/39; A61B 5/00

(52) U.S. Cl. CPC A61B 5/0215; A61B 5/7282; A61B 5/686; A61N 1/36564; A61N 1/3987

(57) ABSTRACT

A medical device system and method for monitoring a cardiovascular pressure signal to identify an atrial arrhythmia that includes a sensor sensing a cardiovascular pressure signal and a pressure analysis module that is configured to determine at least one of an interval dispersion and an amplitude dispersion of the sensed pressure signal, compare the at least one of an interval dispersion and an amplitude dispersion of the sensed pressure signal to a dispersion threshold, and determine whether the atrial arrhythmia is occurring in response to the comparing

### FIELD OF THE DISCLOSURE

The disclosure relates generally to medical devices and, in particular, to atrial arrhythmia detection in an implantable medical device and implantable medical device system.

### BACKGROUND

Implantable medical sensors are used for sensing physiological signals in a patient for use in diagnosing a cardiac disease state or managing patient cardiac therapies. A pressure sensor positioned in the heart or in a blood vessel, such as the pulmonary artery, for example, is highly useful in monitoring cardiovascular conditions, including heart failure or hypertension, by measuring heart rate through the sensing of pressure pulses generated by the ventricular contraction of a patient's heart. For example, a capacitive pressure sensor includes one capacitor electrode along a diaphragm and a second capacitor electrode substantially parallel to and held a few micrometers from the electrode of the diaphragm. An “air gap” provides insulation between the two parallel electrodes. As the blood pressure changes, the diaphragm flexes closer to or further away from the second electrode, resulting in a change in capacitance. The capacitance can be measured in many ways and can be converted to pressure using a calibration algorithm.

During normal sinus rhythm (NSR), the heart beat is regulated by electrical signals produced by the sino-atrial (SA) node located in the right atrial wall. Each atrial depolarization signal produced by the SA node spreads across the atria, causing the depolarization and contraction of the atria, and arrives at the atrioventricular (A-V) node. The A-V node responds by propagating a ventricular depolarization signal through the bundle of His of the ventricular septum and thereafter to the bundle branches and the Purkinje muscle fibers of the right and left ventricles.

Atrial tachyarrhythmia includes the disorganized form of atrial fibrillation and varying degrees of organized atrial tachycardia, including atrial flutter. Atrial fibrillation (AF) occurs because of multiple focal triggers in the atrium or because of changes in the substrate of the atrium causing heterogeneities in conduction through different regions of the atria. The ectopic triggers can originate anywhere in the left or right atrium or pulmonary veins. The AV node will be bombarded by frequent and irregular atrial activations but will only conduct a depolarization signal when the AV node is not refractory. The ventricular cycle lengths will be irregular and will depend on the different states of refractoriness of the AV-node.

In the past, atrial arrhythmias have been largely undertreated due to the perception that these arrhythmias are relatively benign. As more serious consequences of persistent atrial arrhythmias have come to be understood, such as an associated risk of relatively more serious ventricular arrhythmias and stroke, there is a growing interest in monitoring and treating atrial arrhythmias.

Current pressure sensors have been employed to detect cardiovascular conditions such as atrial fibrillation using heart rate, pulmonary artery systolic pressure (PASP) and pulmonary artery diastolic pressure (PADP). U.S. Patent Publication No. 2013/0204147 to Blomqvist et. al., for example, teaches detecting atrial fibrillation based on pulmonary artery pressure (PAP) data, such as cycle-to-cycle variations of one or more parameters derived from the PAP data. While the more serious consequences associated with persistent atrial fibrillation are becoming evident, treatments for slowing or terminating persistent atrial fibrillation tends to be very difficult. As a result, many treatments are typically utilized, alone or in combination, such as ablation therapy, ingestion of certain specific medications, or changes in dosage of medication, including antiarrhythmic agents like, flecainide or amiodarone, for example, or oral anticoagulants such as dabigatran, rivaroxaban and apixaban. Therefore, what is needed is a method for improving detection of persistent atrial fibrillation to assist in determining treatment of atrial fibrillation and the effectiveness of such treatment(s).

### BRIEF DESCRIPTION OF THE DRAWINGS

FIG. 1 is a schematic diagram of an exemplary pressure sensor positioned within a patient's heart, according to an embodiment of the disclosure.

FIG. 2 is a functional block diagram illustrating an exemplary configuration of a pressure sensor that may be used to implement certain techniques of the disclosure.

FIG. 3A is a perspective view of a pressure sensor according to an embodiment of the present disclosure.

FIG. 3B is a perspective view of a pressure sensor according to another embodiment of the present disclosure.

FIG. 4 is a top plan view of the sensing module of FIG. 3A.

FIG. 5 is a flowchart of detecting an atrial arrhythmia using a pressure signal in a medical device, according to an embodiment of the present disclosure.

FIGS. 6A and 6B are graphical representations of the determination of a dispersion pattern associated with sensed pressure pulses of a pressure signal for detecting an atrial arrhythmia, according to an embodiment of the present disclosure.

FIG. 6C is a flowchart of a method for determining a cardiac event, according to an embodiment of the present disclosure.

FIG. 6D is a flowchart of a method for determining a cardiac event, according to an embodiment of the present disclosure.

FIG. 7 is a flowchart of detecting an atrial arrhythmia using a pressure signal in a medical device, according to an embodiment of the present disclosure.

FIG. 8 is a graphical representation of detecting an atrial arrhythmia using a pressure signal in a medical device, according to an embodiment of the present disclosure.

### DETAILED DESCRIPTION

In the following description, references are made to illustrative embodiments. It is understood that other embodiments may be utilized without departing from the scope of the disclosure.

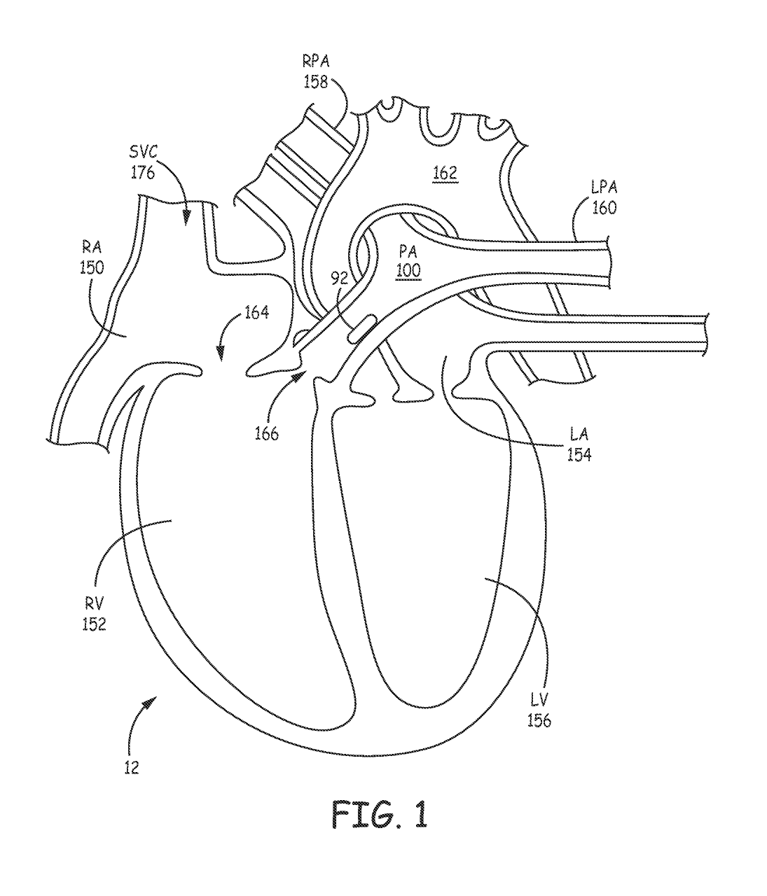

FIG. 1 is a schematic diagram of an exemplary pressure sensor positioned within a patient's heart, according to an embodiment of the disclosure. Exemplary pressure sensors that may be utilized in the present disclosure are described, for example, in commonly assigned U.S. Pat. No. 9,131,858 to Flo et al., and in commonly assigned U.S. Patent Publication No. 2012/0277600 to Greenhut, both incorporated by reference herein in their entireties. As illustrated in FIG. 1, a heart 12 includes a pulmonary artery 100, a right atrium 150, a right ventricle 152, a left atrium 154, a left ventricle 156, a right pulmonary artery 158, a left pulmonary artery 160, an aorta 162, an atrioventricular valve 164, a pulmonary valve 166, an aortic valve 168, and a superior vena cava 176. A pressure sensor 92 may, as shown in FIG., be placed inside the pulmonary artery 100 of the heart 12. In some example implementations, sensor 92 may be placed within the main pulmonary artery 100, the right pulmonary artery 158 or any of its branches, and/or within the left pulmonary artery 160 or any of its branches, or within the right ventricle. In other example implementations, multiple pressure sensors 92 may be placed at various locations within the pulmonary artery 100, the right pulmonary artery 158 or any of its branches, and/or the left pulmonary artery 160 or any of its branches.

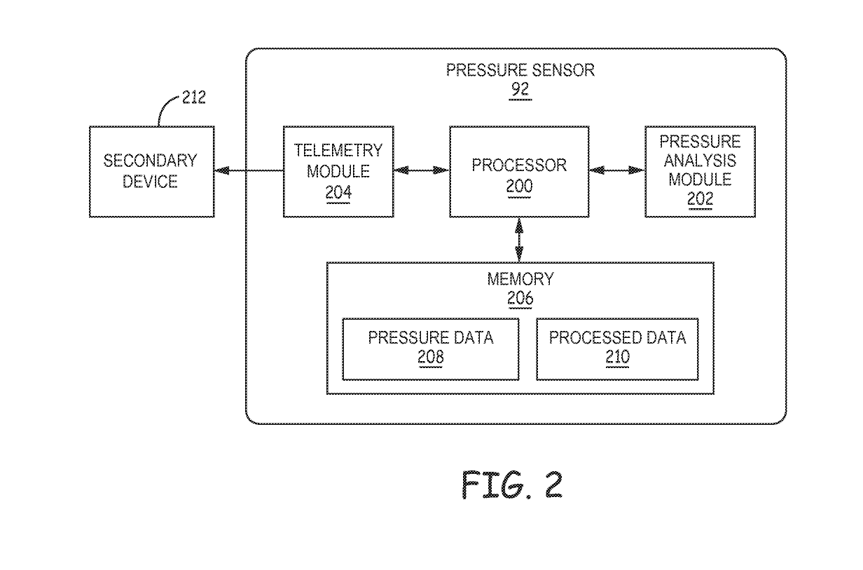

As illustrated in FIG. 1, the pressure sensor 92 may be a leadless assembly, e.g., need not be coupled to an IMD or other device via a lead, and need not otherwise be coupled to any leads. Although not depicted, pressure sensor 92 may include wireless communication capabilities such as low frequency or radiofrequency (RF) telemetry, as well other wireless communication techniques that allow sensor 92 to communicate with an external device 212 (shown in FIG. 2), external to the sensor 92, such as an implantable medical device (IMD) or a programmer, for example. The pressure sensor 92 may be affixed to the wall of the pulmonary artery or the wall of the right ventricle using any number of well-known techniques. For example, the pressure sensor 92 may include fixation elements, e.g., helical tines, hooked tines, barbs, or the like, that allow the sensor 92 to be secured to the pulmonary artery 100. In other examples, the pressure sensor 92 may be attached to a stent having any variety of conformations, for example, and the stent/sensor combination may be implanted within the pulmonary artery 100.

The pressure sensor 92 may be implanted within the pulmonary artery 100, for example, using a delivery catheter. For example, a physician may deliver the pressure sensor(s) 92 via a delivery catheter, transvenously through either the internal jugular or femoral veins. The delivery catheter then extends through the superior vena cava 176, the right atrioventricular valve 164, the right ventricle 152, and the pulmonary valve 166 into the pulmonary artery 100. In other examples, the pressure sensor 92 may be implanted after a physician has opened the patient's chest by cutting through the sternum.

The pressure sensor 92 generates pressure information representing a pressure signal as a function of the fluid pressure in the pulmonary artery 100, for example, that is utilized to detect an atrial arrhythmia, such as atrial fibrillation, as described in more detail below. In response to the atrial arrhythmia detection algorithm described below, the pressure sensor 92 generate an alarm or transmit associated data to a device 24 external to the sensor 92, such as an implantable medical device (not shown), a programmer, and/or another device, e.g., external monitoring equipment, which may receive, monitor, and analyze the pressure information, as will be described in more detail below.

FIG. 2 is a functional block diagram illustrating an exemplary configuration of a pressure sensor that may be used to implement certain techniques of the disclosure. As illustrated in FIG. 2, the pressure sensor 92 includes a processor 200, pressure analysis module 202, telemetry module 204, and memory 206. Processor 200 may store pressure information as pressure data 208 in memory 206. Pressure data 208 may include raw, unprocessed pressure information that represents a pressure signal within a pulmonary artery of a patient. In some examples, telemetry module 204 may transmit pressure data 208 to an external device 212, external to the pressure sensor 92, such as an implantable medical device or monitoring device, or an external monitor or programmer, for example, for further analysis.

The pressure analysis module 202 processes pressure information sensed by pressure sensor 92 and stores the processed information in memory 206 as processor data 210. Pressure analysis module 202 may be implemented as software, firmware, hardware or any combination thereof. In some example implementations, pressure analysis module 202 may be a software process implemented in or executed by processor 200. Processed data 210 may represent the values determined based on pressure data 208, such as systolic pressure data, and diastolic pressure data as processed and/or determined by pressure analysis module 202. The telemetry module 204 may transmit processed data 210 to the external device 212, such as an implantable medical device, programmer, or another external device, e.g., for further analysis.

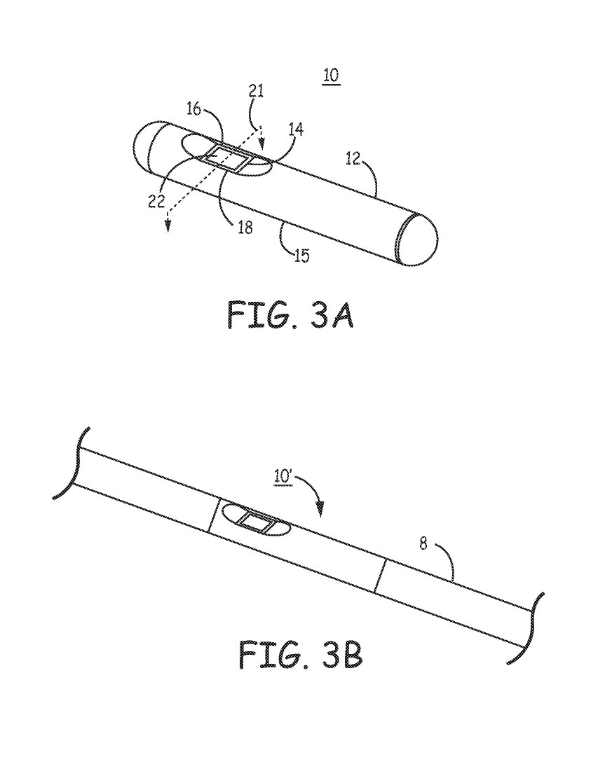

FIG. 3A is a perspective view of a pressure sensor according to an embodiment of the present disclosure. As illustrated in FIG. 3A, a pressure sensor 92 includes a housing 12 enclosing a sensor transducer and associated circuitry (not shown in FIG. 3A). The housing 12 includes a substantially flat portion 14 extending between a first outer side 16 and a second outer side 18. A flexible diaphragm 22 extends along substantially flat portion 14 between outer sides 16 and 18. Diaphragm 22 is exposed to external pressures applied to the outer surface of flat portion 14. In the embodiment shown, the housing 12 is substantially cylindrical and thus includes a rounded or curved wall 15 opposing the substantially flat portion 14. In other embodiments, the housing may be configured as other rounded, prismatic, or geometric shapes and may or may not be elongated, but generally includes a substantially flat portion along which the sensor diaphragm extends and a curved or substantially flat side opposing the flat portion.

Pressure sensor 92 is shown as a wireless sensor which may be implanted within the blood stream or blood volume or at any extravascular location targeted for monitoring a physiological signal. Pressure sensor 92 may include fixation elements or members attached to housing 12 to facilitate fixation of pressure sensor 92 at a desired implant site. Fixation members are not explicitly shown in FIG. 3A, but it is recognized that various embodiments may include any type of fixation members used to anchor implantable medical devices at an anatomical location.

FIG. 3B is a perspective view of a pressure sensor according to another embodiment of the present disclosure. As illustrated in FIG. 3B, in other embodiments, as shown in FIG. 3B, a pressure sensor 92′ may be carried by an elongated flexible medical lead body 8. Pressure sensor 92′ may correspond generally to pressure sensor 92 shown in FIG. 3A with the exception of being coupled to the lead body 8 and may be configured for wireless or wired signal transmission to an associated medical device. Elongated lead body 8 may additionally carry other sensors or electrodes and typically includes elongated electrical conductors extending between sensors and/or electrodes carried by the lead body 8 and a proximal electrical connector assembly (not shown). The connector assembly is adapted for connection to a medical device such as a pacemaker, cardioverter/defibrillator, neurostimulator, monitoring device or the like to provide electrical connection between sensors and/or electrodes carried by the lead body 8 and the associated medical device.

Examples of implantable devices within which the sensor module may be utilized, in either a wireless configuration as shown in FIG. 1A or a lead-based configuration as shown in FIG. 1B, are generally disclosed in commonly-assigned U.S. Pat. No. 5,540,731 (Testerman), U.S. Pat. No. 7,367,951 (Bennett), U.S. Pat. No. 6,580,946 (Struble), and U.S. Publication No. 2009/0299429 (Mayotte), for example, all of which references are incorporated herein by reference in their entirety. A capacitive pressure sensor is generally disclosed in commonly-assigned U.S. Pat. No. 5,535,752 (Halperin), hereby incorporated herein by reference in its entirety.

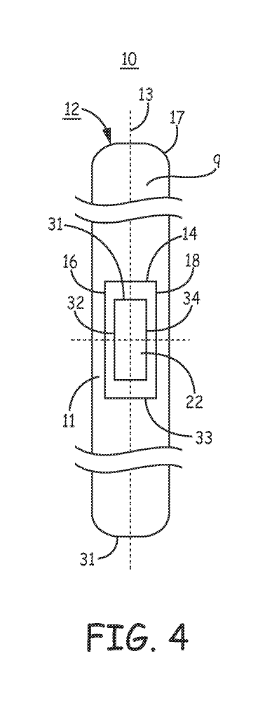

FIG. 4 is a top plan view of the sensing module of FIG. 3A. As illustrated in FIG. 4, housing 12 includes an outer wall 9 having a cut-out portion 11 formed in the outer wall 9. The cut-out portion 11 is formed having a first longitudinal outer side 16 and a second longitudinal outer side 18, both extending longitudinally along the pressure sensor 92, and a first lateral outer side 23 and a second lateral outer side 25 extending laterally along the pressure sensor 92 between the first outer side 16 and the second outer side 18. A substantially flat portion 14 is formed within the cut-out portion 11 along which diaphragm 22 extends. Diaphragm 22 has opposing longitudinal edges 32 and 34 separated by a width of the diaphragm 22 and defined by opposing lateral edges 31 and 33. Opposing longitudinal edges 32 and 34 extend adjacent to, and in relative close proximity of, outer sides 16 and 18 of cut-out portion 11 of housing 12. Longitudinal edges 32 and 34 extend substantially parallel with the longitudinal axis 13 of the elongated pressure sensor 92. In the embodiment shown, longitudinal axis 13 also defines a medial plane between diaphragm edges 32 and 34, although diaphragm 22 is not necessarily centered on a central longitudinal axis of the pressure sensor 92 in all embodiments. Outer sides 16 and 18 extend substantially parallel to diaphragm edges 32 and 34, respectively. Outer sides 16 and 18 extend between housing ends 17 and 19.

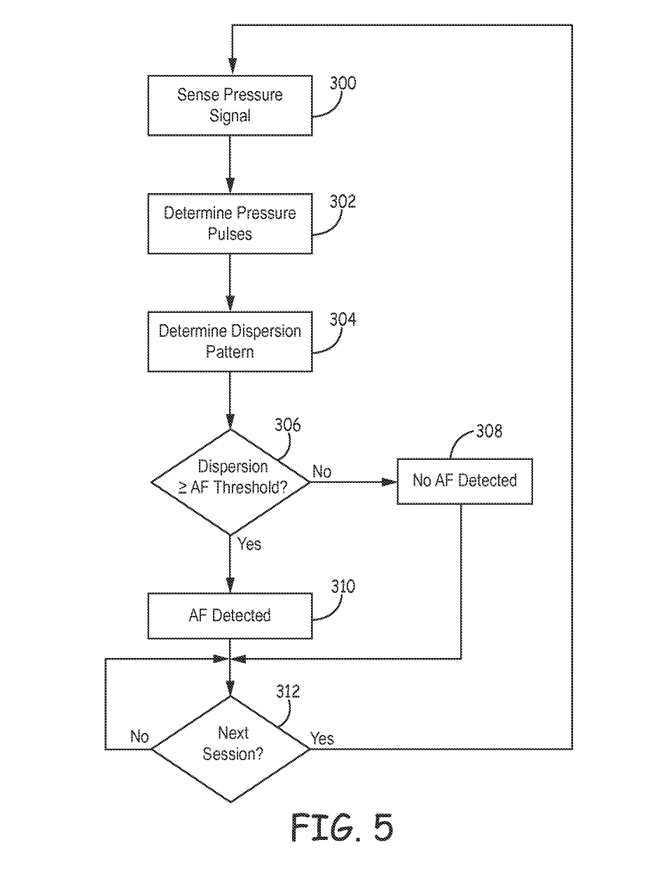

FIG. 5 is a flowchart of detecting an atrial arrhythmia using a pressure signal in a medical device, according to an embodiment of the present disclosure. As illustrated in FIG. 5, according to an embodiment of the present disclosure, during the detection or confirmation of the occurrence of atrial fibrillation, the pressure sensor 92 senses a pressure signal to generate pressure data, Block 300, during predetermined scheduled daily data collection sessions. For example, data collection sessions may be programmed to be performed a certain number of times or sessions per day, such as 8 sessions per day (one data collection session every three hours). The pressure data collected from the pressure signal sensed during the pressure data collection session is analyzed by the pressure sensor 92 via the pressure analysis module 202 to determine pressure pulses associated with the contraction of the patient's heart, Block 302. A dispersion pattern associated with the regularity of the determined pressure pulses is determined, Block 304, and a determination is made as to whether the dispersion pattern is associated with an atrial arrhythmia, such as atrial fibrillation for example, by comparing the dispersion pattern to a dispersion threshold, Block 306, as described below. If the dispersion pattern is determined to be less than the dispersion threshold, No in Block 306, the data collection session is identified as not being associated with atrial fibrillation event, Block 308. If the dispersion pattern is determined not to be less than the dispersion threshold, i.e., greater than or equal to the dispersion threshold, Yes in Block 306, the data collection session is identified as being associated with atrial fibrillation event, Block 310. Once the current data collection session has been identified as being either an atrial fibrillation event, Block 308, or not an atrial fibrillation event, Block 310 based on the determined variability or dispersion pattern, the device waits a predetermined time period for the next scheduled data collection session to occur, Block 312, i.e., three hours for example. Once the next scheduled data collection session is scheduled to occur, Yes in Block 312, the process is repeated for the next data collection session. While the length of each data collection session is programmable, according to one embodiment each data collection session occurs for 16 seconds, for example.

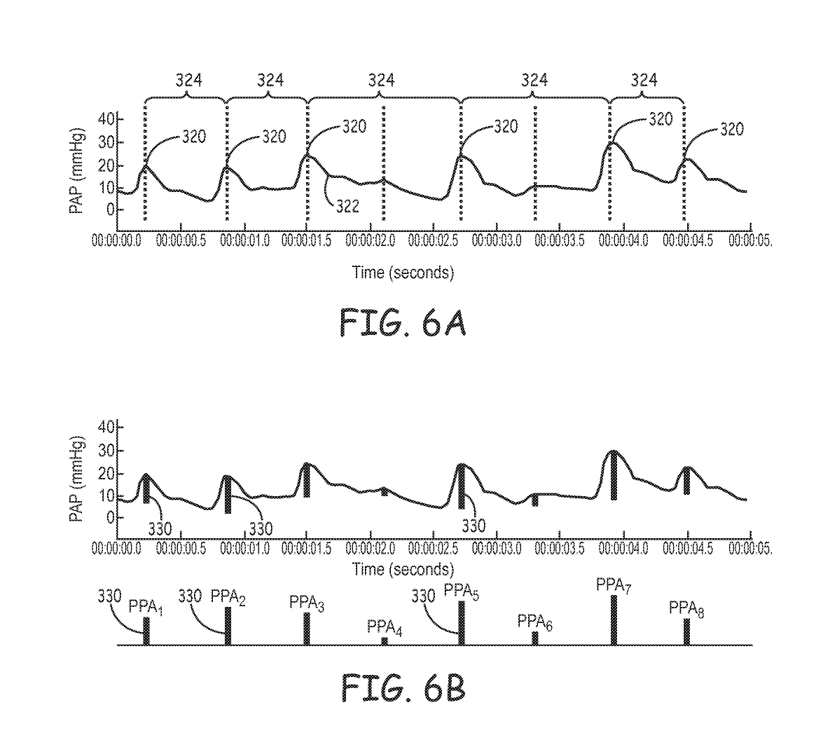

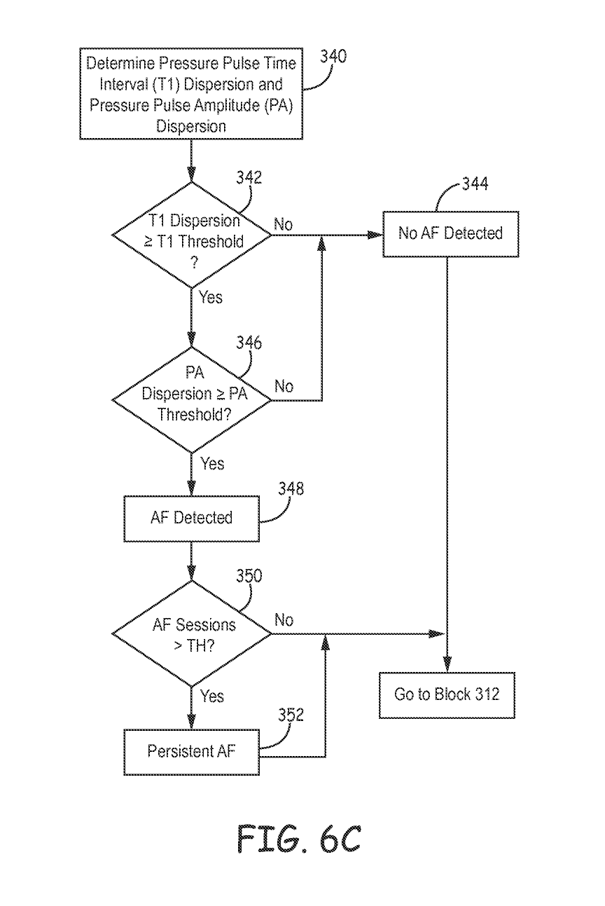

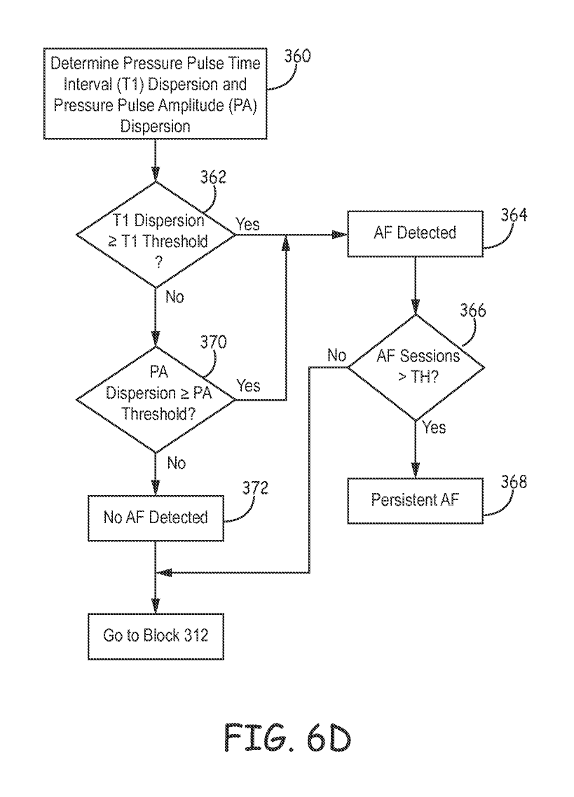

FIGS. 6A and 6B are graphical representations of the determination of a dispersion pattern associated with sensed pressure pulses of a pressure signal for detecting an atrial arrhythmia, according to an embodiment of the present disclosure. As illustrated in FIG. 6A, according to one embodiment, in order to detect the occurrence of atrial fibrillation, for example, peak systolic pressure points 320 of the sensed pressure signal 322 are identified by the sensor device 92 during a data collection session, and a dispersion pattern is determined based on the variability that exists between time intervals 324 extending between a given peak systolic pressure point (n) and a previous peak systolic pressure point (n-1). The dispersion pattern or variability between the intervals 324 may be determined using known interval variability determination schemes, such as through the use of a Lorentz scatter plot, as generally described in U.S. Pat. No. 7,031,765 to Ritscher et al. and U.S. Pat. No. 8,437,851 to Corbucci et al., both incorporated herein by reference in their entireties. Other methods of determining interval variation are generally disclosed by Sarkar, et al. in U.S. Pat. No. 7,623,911 and in U.S. Pat. No. 7,537,569 and by Houben in U.S. Pat. No. 7,627,368, all of which patents are also incorporated herein by reference in their entireties. The determined dispersion, or variability between the peak pressure point time intervals 324 for the data collection session is compared to a predetermined threshold, and atrial fibrillation is not determined to be present for the session if the dispersion is small, i.e., not greater than the dispersion threshold.

According to one embodiment, the dispersion associated with the difference between pressure pulse intervals 324 may be determined by plotting consecutive pressure pulse intervals against most recent pressure pulse intervals PPIₙ, (PPIₙ₋1) on a Lorentz scatter plot, and determining whether a percentage of the plotted consecutive pressure pulse intervals are outside a given distance from the origin (0,0) of the scatter plot. For example, according to one embodiment, atrial fibrillation is determined to occur if more than 50 percent of the plotted consecutive pressure pulse intervals PPIₙ, (PPIₙ₋₁) are greater than 200 ms from the origin (0,0) of the scatter plot. On the other hand, atrial fibrillation is determined not to be present if the number of plotted consecutive pressure pulse intervals PPIₙ, (PPIₙ₋₁) that are greater than 200 ms from the origin (0,0) of the scatter plot is 50 percent or less.

In some instances, a prolonged ventricular contraction due to non-conducted atrial electrical activation associated with an atrial fibrillation event may cause the next ventricular contraction to be more forceful (due to the potential effect), thus generating an increased pulmonary artery pressure. Therefore, as illustrated in FIG. 6B, according to another embodiment, in order to account for these atrial fibrillation induced pulmonary artery changes for use as an indication of an atrial fibrillation event being detected, a pressure pulse amplitude 330 is determined by the sensor device 92 for each detected cardiac cycle occurring during a data collection session. For example, the sensor device 92 determines the difference (PASP-PADP) between the pulmonary artery systolic pressure (PASP) and the pulmonary artery diastolic pressure (PADP) during each cardiac cycle. A dispersion pattern is determined based on the variability or dispersion that exists between a given pressure pulse amplitude (n) and a previous pressure pulse amplitude (n-1). The dispersion pattern or variability between the pressure pulse amplitudes 330 may be determined using known interval variability determination schemes, such as through the use of a Lorentz scatter plot, as generally described, for example, in U.S. Pat. No. 7,031,765 to Ritscher et al. and U.S. Pat. No. 8,437,851 to Corbucci et al., both incorporated by reference herein in their entireties. Other methods of determining interval variation are generally disclosed by Sarkar, et al. in U.S. Pat. No. 7,623,911 and in U.S. Pat. No. 7,537,569 and by Houben in U.S. Pat. No. 7,627,368, all of which patents are also incorporated herein by reference in their entirety. The determined dispersion, or variability between the pressure pulse amplitudes 330 for the data collection session is compared to a predetermined threshold, and atrial fibrillation is not determined to be present for the session if the dispersion is small, i.e., not greater than the dispersion threshold.

According to one embodiment, the dispersion associated with the difference between pressure pulse amplitudes 330 may be determined by plotting consecutive pressure pulse amplitudes against most recent pressure pulse amplitudes PPAₙ, (PPAₙ₋₁) on a Lorentz scatter plot, and determining whether a percentage of the plotted consecutive pressure pulse amplitudes are outside a given distance from the origin (0,0) of the scatter plot. For example, according to one embodiment, atrial fibrillation is determined to occur if more than 50 percent of the plotted consecutive pressure pulse amplitudes PPAₙ, (PPAₙ₋₁) are greater than 200 ms from the origin (0,0) of the scatter plot. On the other hand, atrial fibrillation is determined not to be present if the number of plotted consecutive pressure pulse amplitudes PPAₙ, (PPAₙ₋₁) that are greater than 200 ms from the origin (0,0) of the scatter plot is 50 percent or less.

Returning to FIG. 5, according to an embodiment of the present disclosure, during the determination of the pressure pulses Block 302 and the associated dispersion pattern Block 304, the sensor device 92 may determine whether an atrial fibrillation event is occurring by comparing one or both of the dispersion of the time intervals 324, and the dispersion of the pressure pulse amplitudes 330, to a dispersion threshold. In this way, the sensor device 92 may determine whether the dispersion pattern is indicative of an atrial arrhythmia event, Block 306, based on one of either the dispersion associated with the time intervals 324 of the pressure pulse amplitudes determined during each data collection session, or the dispersion associated with the pressure pulse amplitudes 330 determined during each data collection session, or based on both the dispersion associated with the time intervals 324 of the pressure pulse amplitudes and the dispersion associated with the pressure pulse amplitudes 330 determined during each data collection session.

FIG. 6C is a flowchart of a method for determining a cardiac event, according to an embodiment of the present disclosure. For example, as illustrated in FIG. 6C, according to one embodiment, during the determination as to whether the data collection session is associated with an atrial fibrillation event, Blocks 306-310 of FIG. 5, both a pressure pulse time interval (TI) dispersion and a pressure pulse amplitude (PA) dispersion is determined, Block 340, as described above, and a determination is made as to whether the time interval dispersion is greater than a time interval dispersion threshold, Block 342. If the time interval dispersion is not greater than the time interval dispersion threshold, No in Block 342, atrial fibrillation is not determined to occur for the current data collection session, Block 344, and the device waits the predetermined time period for the next scheduled data collection session to occur, Block 312 of FIG. 5.

If the time interval dispersion is greater than or equal to the time interval dispersion threshold, Yes in Block 342, a determination is made as to whether the pulse amplitude dispersion is greater than a pulse amplitude dispersion threshold, Block 346. If the pulse amplitude dispersion is not greater than the pulse amplitude dispersion threshold, No in Block 346, atrial fibrillation is not determined to occur for the current session, Block 344, and the device waits the predetermined time period for the next scheduled data collection session to occur, Block 312 of FIG. 5. If the pulse amplitude dispersion is greater than or equal to the pulse amplitude dispersion threshold, Yes in Block 346, atrial fibrillation is determined to occur for the current data collection session, Block 348. A determination is made as to whether the number of data collection sessions for which atrial fibrillation is determined to occur exceeds an AF sessions threshold, Block 350. If the number of data collection sessions for which atrial fibrillation is determined to occur exceeds the AF sessions threshold, Yes in Block 350, persistent atrial fibrillation is determined to occur, Block 352, and the device waits the predetermined time period for the next scheduled data collection session to occur, Block 312 of FIG. 5. The sensor device 90 may store the determination of persistent atrial fibrillation occurring in Block 352, which information may then be subsequently transmitted to a programming device and subsequently used by the programming device to adjust a treatment, such as ablation therapy, a pacing therapy, ingestion of certain specific medications, or to adjust a dosage amount of a medication, for example.

According to another embodiment, the identification of persistent atrial fibrillation may be transmitted from the sensor 92 to the external device, which may include an implantable medical device, such as an implantable cardioverter defibrillator as described, for example, in commonly assigned U.S. Patent Publication No. 2012/0277600 to Greenhut, or a subcutaneously implanted device, such as a monitoring device, as described in commonly assigned U.S. Patent Application No. 61/199,424, to Ghosh e al., or an implantable cardiac defibrillator coupled to an extravascular lead, as described for example in commonly assigned U.S. patent application Ser. No. 14/801/049 to Ghosh et. al., al incorporated herein by reference in their entireties.

According to one embodiment, the AF sessions threshold may be set as a predetermined number of sessions of the totally daily number of sessions, such as 6 out of the eight daily data collection sessions, for example. In one embodiment, the six out of eight sessions may overlap between consecutive days, so that for example, the last two sessions of one day result in atrial fibrillation being detected, along with 4 of the next six sessions from the next day. According to another embodiment, the AF sessions threshold may be set as being three consecutive sessions being determined as atrial fibrillation, either in a single day or in two consecutive days (i.e., the last session of one day and the first two sessions of the next day, for example). In yet another embodiment, the AF sessions threshold may be set as being satisfied if either a predetermined number of sessions of the totally daily number of sessions are determined as being associated with atrial fibrillation, or if three consecutive sessions are determined as being associated with atrial fibrillation. In this way, both the dispersion associated with the time intervals 324 of the pressure pulse amplitudes and the dispersion associated with the pressure pulse amplitudes 330 determined during each data collection session must be greater than respective thresholds in order for atrial fibrillation to be detected for the data collection session.

FIG. 6D is a flowchart of a method for determining a cardiac event, according to an embodiment of the present disclosure. As illustrated in FIG. 6D, according to another embodiment, during the determination as to whether the data collection session is associated with an atrial fibrillation event, Blocks 306-310 of FIG. 5, both a pressure pulse time interval (TI) dispersion and a pressure pulse amplitude (PA) dispersion is determined, Block 360, as described above, and a determination is made as to whether the time interval dispersion is greater than a time interval dispersion threshold, Block 362. If the time interval dispersion is greater than the time interval dispersion threshold, Yes in Block 362, atrial fibrillation is determined to occur for the current data collection session, Block 364. A determination is made as to whether the number of data collection sessions for which atrial fibrillation is determined to occur exceeds an AF sessions threshold, Block 366, as described above, so that persistent atrial fibrillation is determined to occur. In response to determining persistent atrial fibrillation has occurred, Block 368, the device stores the determination for use as described above, and waits the predetermined time period for the next scheduled data collection session to occur, Block 312 of FIG. 5.

If the time interval dispersion is not greater than the time interval dispersion threshold, No in Block 362, a determination is made as to whether the pulse amplitude dispersion is greater than or equal to a pulse amplitude dispersion threshold, Block 370, as described above. If the pulse amplitude dispersion is not greater than or equal to the pulse amplitude dispersion threshold, No in Block 370, atrial fibrillation is not determined to occur for the current session, Block 372, and the device waits the predetermined time period for the next scheduled data collection session to occur, Block 312 of FIG. 5. If the pulse amplitude dispersion is greater than or equal to the pulse amplitude dispersion threshold, Yes in Block 370, atrial fibrillation is determined to occur for the current data collection session, Block 364. A determination is made as to whether the number of data collection sessions for which atrial fibrillation is determined to occur exceeds an AF sessions threshold, Block 366, as described above. If the number of data collection sessions for which atrial fibrillation is determined to occur exceeds the AF sessions threshold, Yes in Block 366, persistent atrial fibrillation is determined to occur, Block 368, and the sensing device 92 stores the data, for use as described above, and waits the predetermined time period for the next scheduled data collection session to occur, Block 312 of FIG. 5.

In this way, if one of the dispersion associated with the time intervals 324 of the pressure pulse amplitudes and the dispersion associated with the pressure pulse amplitudes 330 determined during each data collection session is determined to be greater than respective thresholds, atrial fibrillation may be detected for the data collection session. It is understood that while the determination of pressure pulse time interval dispersion is illustrated as occurring prior to the determination of the pressure pulse amplitude dispersion, the order of performing the two features may be reversed without departing from the intended present disclosure.

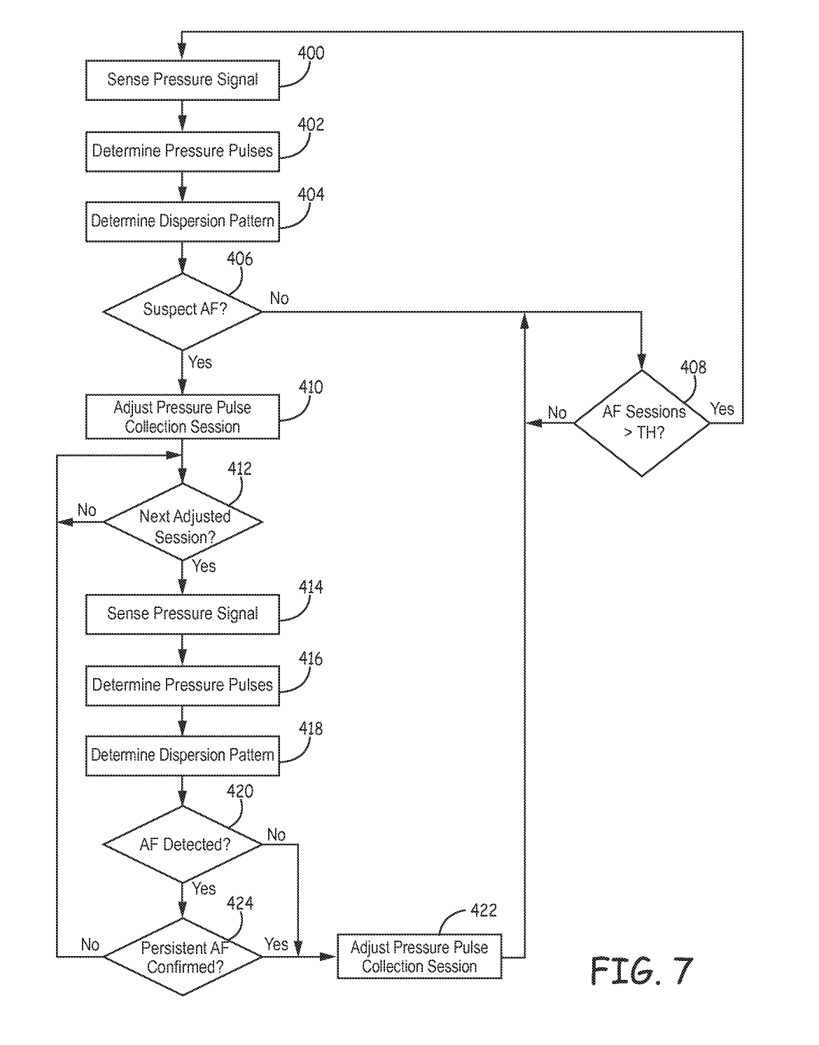

FIG. 7 is a flowchart of detecting an atrial arrhythmia using a pressure signal in a medical device, according to an embodiment of the present disclosure. As illustrated in FIG. 7, according to an embodiment of the present disclosure, in order to detect whether an atrial fibrillation event is occurring, the sensor device 92 senses a pressure signal to generate pressure data, Block 400, during predetermined scheduled daily data collection sessions. For example, data collection sessions may be programmed to be performed a certain number of times or sessions per day, such as 8 sessions per day (one data collection session every three hours). The pressure data collected from the pressure signal sensed during the pressure data collection session is then analyzed by the pressure sensor 92 via the pressure analysis module 202 to determine pressure pulses associated with the contraction of the patient's heart, Block 402. A dispersion pattern associated with the regularity of the determined pressure pulses is determined, Block 404, and a determination is made as to whether an atrial fibrillation event is suspected, Block 406, based on the determined dispersion pattern, as described above. If an atrial fibrillation event is not suspected to be occurring, No in Block 406, the sensor device 92 waits for the next scheduled data collection session to occur, i.e., three hours for example, Block 408. Once the next scheduled data collection session is scheduled to occur, Yes in Block 408, the process, Blocks 400-406, is repeated for the next data collection session.

If an atrial fibrillation event is suspected to be occurring as a result of the determined dispersion pattern, Yes in Block 406, the sensor device 92 adjusts the scheduled pressure data collection sessions, Block 410, in order to enhance the accuracy of the detection of atrial fibrillation by the sensor device 92, Block 408. For example, according to one embodiment, if an atrial fibrillation event is suspected to be occurring, Yes in Block 406, the sensor device 92 increases the number of scheduled pressure data collection sessions from the initial or regularly scheduled number of sessions, i.e., every three hours, to an increased number of sessions, such as every five minutes, for example.

Once the adjusted scheduled session is scheduled to occur, i.e., five minutes expires, an adjusted pressure pulse data collection session is initiated, Yes in Block 412, so that the sensor device 92 senses a pressure signal, Block 414, and determines pressure pulses associated with the sensed pressure signal for the adjusted session, Block 416. A dispersion pattern associated with the regularity of the determined pressure pulses is determined, Block 418, and the sensor device 92 determines whether an atrial fibrillation event is detected for the adjusted session based on the dispersion pattern of the sensed pressure pulses, Block 420, as described above.

If an atrial fibrillation event is not detected, No in Block 420, the sensor device 92 adjusts the scheduled pressure data collection sessions back from the adjusted enhanced number of sessions, i.e., every five minutes, to the initial or regularly scheduled number of sessions, i.e., every three hours, Block 422, and waits for the next scheduled data collection session to occur, i.e., three hours for example, Block 408. Once the next scheduled data collection session is scheduled to occur, Yes in Block 408, the process, Blocks 400-406, is repeated for the next data collection session.

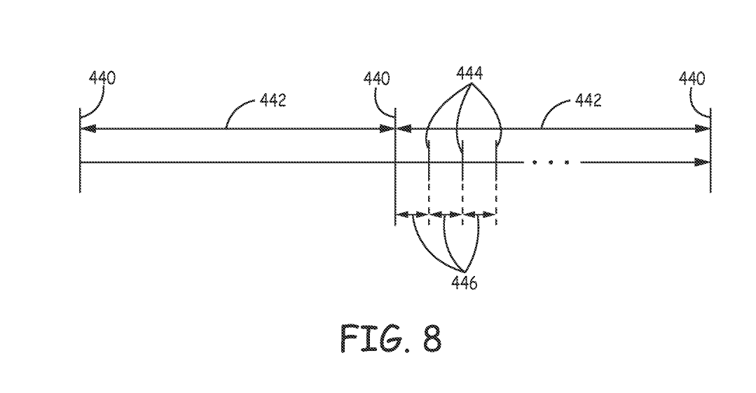

FIG. 8 is a graphical representation of detecting an atrial arrhythmia using a pressure signal in a medical device, according to an embodiment of the present disclosure. As illustrated in FIGS. 7 and 8, according to an embodiment of the present disclosure, the sensor device 92 initially operates to detect whether an atrial event is occurring by sensing a pressure signal to generate pressure data. The pressure signal is sensed during initial scheduled data collection sessions 440 that occur over an initial data collection session frequency, such as eight times per day, or every three hours per day 442, for example, as described above. Once an atrial arrhythmia event, such as atrial fibrillation, is detected during one of the initial scheduled data collection sessions 440, the sensor device 92 adjusts the frequency of the data collection session from the initial frequency of the initial scheduled data collection session 440 to an enhanced frequency of the scheduled data collection session 444, such as every five minutes 446, for example.

If the sensor device 92 determines during the subsequent adjusted data collection session 444 that an atrial fibrillation is not detected, No in Block 420, the sensor device 92 adjusts the frequency of the scheduled data collection sessions from the enhanced adjusted frequency 444 to the initial frequency of data collection 440, Block 440, and the process, Blocks 400-406, is repeated for the next scheduled initial data collection session 440, and so on. If the sensor device 92 determines during the subsequent adjusted data collection session 444 that an atrial fibrillation is detected, Yes in Block 420, the sensor device 92 determines whether persistent atrial fibrillation is confirmed, Block 422. For example, according to one embodiment, the sensor device 92 determines whether atrial fibrillation has been confirmed for a predetermined number of consecutive adjusted data collection sessions 444, such as three consecutive adjusted data collections 444, for example. If atrial fibrillation has not been confirmed for the predetermined number of consecutive adjusted data collection sessions 444, persistent atrial fibrillation is not confirmed, No in Block 422, and the process, Blocks 412-420, is repeated for the next adjusted data collection session 444, and so on.

Once atrial fibrillation has been confirmed for the predetermined number of consecutive adjusted data collection sessions 444, persistent atrial fibrillation is confirmed, Yes in Block 422, and is stored in memory 206 by the sensing device 92. In some examples, telemetry module 204 may transmit pressure data 208 to a monitoring device, such as an implantable medical device, an external monitor or programmer, for example, for further analysis. For example, the information may be utilized to make adjustments to a delivered therapy, to a patient's medication dosage regime, or to determine whether a new or additional medication may be recommended.

The various features described herein and shown in the accompanying drawings may be used alone or in any combination to reduce contact pressure on a sensor diaphragm. Thus, housings for medical sensor modules have been presented in the foregoing description with reference to specific embodiments. It is appreciated that various modifications to the referenced embodiments may be made without departing from the scope of the disclosure as set forth in the following claims.

We claim:

1. A medical device system for monitoring a cardiovascular pressure signal to identify an atrial arrhythmia, comprising:
a sensor sensing a cardiovascular pressure signal;
a pressure analysis module configured to determine at least one of an interval dispersion and an amplitude dispersion of the sensed pressure signal, compare the at least one of an interval dispersion and an amplitude dispersion of the sensed pressure signal to a dispersion threshold, and determine whether the atrial arrhythmia is occurring in response to the comparing.

2. The medical device system of claim 1, wherein the pressure analysis module is configured to determine the at least one of an interval dispersion and an amplitude dispersion of the sensed pressure signal for each of a first plurality of data collection sessions, compare a number of data collection sessions of the first plurality of data collection sessions for which the atrial arrhythmia is determined to occur to a persistent atrial arrhythmia threshold, and identify a persistent atrial arrhythmia in response to the number of data collection sessions of the first plurality of data collection sessions for which the atrial arrhythmia is determined to occur satisfying the persistent atrial arrhythmia threshold.

3. The medical device system of claim 2, wherein the first plurality of data collection sessions comprises eight data collection sessions per day and identifying a persistent atrial fibrillation in response to determining the atrial arrhythmia is occurring for six data collection sessions of the eight data collection sessions.

4. The medical device system of claim 2, wherein the pressure analysis module is configured to adjust the first plurality of data collection sessions to an adjusted number of data collections per day in response to the atrial arrhythmia being determined for a data collection session of the adjusted plurality of data collection sessions.

5. The medical device system of claim 4, wherein the first plurality of data collection sessions comprises eight data collection sessions per day and the adjusted plurality of data collection sessions comprises more than eight collection sessions per day.

6. The medical device system of claim 5, wherein the pressure analysis module is configured to identify a persistent atrial arrhythmia in response to the atrial arrhythmia being determined for three consecutive data collection sessions of the adjusted plurality of data collection sessions.

7. The medical device system of claim 4, wherein the pressure analysis module is configured to adjust the data collection sessions from the adjusted plurality of data collection sessions to the first plurality of data collection sessions in response to an arrhythmia not being determined for one data collection session of the adjusted data collections sessions.

8. A medical device system for monitoring a cardiovascular pressure signal to identify an atrial arrhythmia, comprising:
a sensor sensing a cardiovascular pressure signal;
a pressure analysis module configured to determine at least one of an interval dispersion and an amplitude dispersion of the sensed pressure signal, compare the at least one of an interval dispersion and an amplitude dispersion of the sensed pressure signal to a dispersion threshold, and determine whether the atrial arrhythmia is occurring in response to the comparing;
an implantable medical device to monitor a cardiac signal; and
a telemetry module to transmit the determination as to whether the atrial arrhythmia is occurring from the sensor to the implantable medical device, wherein an atrial arrhythmia therapy is adjusted by the implantable medical device in response to the transmitted determination.

9. The medical device system of claim 8, wherein the arrhythmia therapy comprises one of an ablation therapy, a pacing therapy, or ingestion of a medication.

10. The medical device system of claim 8, wherein the implantable medical device is one of an implantable cardioverter defibrillator, a subcutaneously implantable monitoring device, or an implantable cardiac defibrillator coupled to an extravascular lead.

11. The medical device system of claim 8, wherein the pressure analysis module is configured to determine the at least one of an interval dispersion and an amplitude dispersion of the sensed pressure signal for each of a first plurality of data collection sessions, compare a number of data collection sessions of the first plurality of data collection sessions for which the atrial arrhythmia is determined to occur to a persistent atrial arrhythmia threshold, and identify a persistent atrial arrhythmia in response to the number of data collection sessions of the first plurality of data collection sessions for which the atrial arrhythmia is determined to occur satisfying the persistent atrial arrhythmia threshold.

12. The medical device system of claim 11, wherein the first plurality of data collection sessions comprises eight data collection sessions per day and identifying a persistent atrial fibrillation in response to determining the atrial arrhythmia is occurring for six data collection sessions of the eight data collection sessions.

13. The medical device system of claim 11, wherein the pressure analysis module is configured to adjust the first plurality of data collection sessions to an adjusted number of data collections per day in response to the atrial arrhythmia being determined for a data collection session of the adjusted plurality of data collection sessions.

14. The medical device system of claim 13, wherein the first plurality of data collection sessions comprises eight data collection sessions per day and the adjusted plurality of data collection sessions comprises more than eight collection sessions per day.

15. The medical device system of claim 14, wherein the pressure analysis module is configured to identify a persistent atrial arrhythmia in response to the atrial arrhythmia being determined for three consecutive data collection sessions of the adjusted plurality of data collection sessions.

16. The medical device system of claim 13, wherein the pressure analysis module is configured to adjust the data collection sessions from the adjusted plurality of data collection sessions to the first plurality of data collection sessions in response to an arrhythmia not being determined for one data collection session of the adjusted data collections sessions.

17. A method of monitoring a cardiovascular pressure signal to identify an atrial arrhythmia, comprising:
sensing a cardiovascular pressure signal;
determining at least one of an interval dispersion and an amplitude dispersion of the sensed pressure signal;
comparing the at least one of an interval dispersion and an amplitude dispersion of the sensed pressure signal to a dispersion threshold; and
determining whether the atrial arrhythmia is occurring in response to the comparing.

18. The method of claim 17, further comprising;
determining the at least one of an interval dispersion and an amplitude dispersion of the sensed pressure signal for each of a first plurality of data collection sessions;
comparing a number of data collection sessions of the first plurality of data collection sessions for which the atrial arrhythmia is determined to occur to a persistent atrial arrhythmia threshold; and
identifying a persistent atrial arrhythmia in response to the number of data collection sessions of the first plurality of data collection sessions for which the atrial arrhythmia is determined to occur satisfying the persistent atrial arrhythmia threshold.

19. The method of claim 18, wherein the first plurality of data collection sessions comprises eight data collection sessions per day and identifying a persistent atrial fibrillation in response to determining the atrial arrhythmia is occurring for six data collection sessions of the eight data collection sessions.

20. The method of claim 18, further comprising adjusting the first plurality of data collection sessions to an adjusted number of data collections per day in response to the atrial arrhythmia being determined for a data collection session of the adjusted plurality of data collection sessions.

21. The method of claim 20, wherein the first plurality of data collection sessions comprises eight data collection sessions per day and the adjusted plurality of data collection sessions comprises more than eight collection sessions per day.

22. The method of claim 21, further comprising identifying a persistent atrial arrhythmia in response to the atrial arrhythmia being determined for three consecutive data collection sessions of the adjusted plurality of data collection sessions.

23. The method of claim 20, further comprising adjusting the data collection sessions from the adjusted plurality of data collection sessions to the first plurality of data collection sessions in response to an arrhythmia not being determined for one data collection session of the adjusted data collections sessions.
